(19) United States
(12) Patent Application Publication
(10) Pub. No.: US 2003/0191423 A1
(43) Pub. Date: Oct. 9, 2003

(54) Breathable non-perforated bandage

(76) Inventor: Robert L. Sun, Succasunna, NJ (US)

Correspondence Address: AUDLEY A. CIAMPORCERO JR., JOHNSON & JOHNSON, ONE JOHNSON & JOHNSON PLAZA, NEW BRUNSWICK, NJ, 08933-7003, US

(21) Appl. No.: 10/405,732

(22) Filed: Apr. 2, 2003

Related U.S. Application Data
(63) Continuation of application No. 09/596,112, filed on Jun. 16, 2000, now abandoned.

(51) Int. Cl. A61F 13/00; A61F 15/00

(52) U.S. Cl. 602/58

(57) ABSTRACT

A non-perforated bandage including: a) a woven fabric backing having a first type of yarn and a second type of yarn, the backing having a first major surface and a second major surface; b) a wound-contacting pad placed on the first major surface of the backing; and c) a hot melt adhesive, wherein the adhesive is applied to the first major surface of the backing material and the bandage is breathable. The yarns differ in their chemical structure or their physical structure (e.g., differing denier) or the nature of their surface treatment.

### BACKGROUND OF THE INVENTION

1. Field of the Invention

The present invention relates to a breathable bandage that does not need to be perforated. The breathable bandage is made from a woven material, which contains yarns of different chemical structures in the warp and fill directions. The bandages comprise hot melt adhesives for adhering the bandages to the skin.

2. Description of the Prior Art

It is well known to apply adhesive bandages to wounds to protect the wound and keep the wound clean. Most commonly, the bandages are made from a polyvinyl chloride (PVC) backing material. One side of the backing material generally contains a centralized pad, which is utilized to keep the wound clean and to cushion the wound. The backing material has adhesive applied thereto. This adhesive secures the pad to the backing and, in use, secures the bandage to the skin of the wearer. A PVC backing material is moisture impermeable and does not allow water vapor to leave the surface of the skin covered by the bandage. This leads to discomfort for the user. In order to overcome this problem, after the bandage backing is coated with adhesive, the backing material may be perforated to allow water vapor to leave the surface of the skin.

Although perforated films are useful, there is a concern that the perforations allow liquid water to reach the surface of the skin and the wound. The presence of water may promote bacterial growth, which can lead to an infection of the wound. Therefore, there is a need for a non-perforated bandage that allows water vapor to evaporate from the surface of the skin under the bandage (i.e. the bandage is “breathable”).

The use of thin breathable films, such as 0.025 mm polyurethane as the backing material for adhesive bandages has been practiced since the 1970s. U.S. Pat. No. 3,645,835 disclosed this type of adhesive dressing for blocking bacteria and liquid water from reaching the wound, but allowing oxygen to penetrate the dressing from the atmosphere and allowing moisture from the skin of the patient to escape from beneath the dressing.

The moisture vapor transmission rate (“MVTR”) indicates the degree of breathability of a given material. In order to obtain the desired MVTR for adhesive bandages and the like wound dressings, these types of films are generally thin, i.e. less than 0.05 mm in thickness.

Because of the nature of the polymers used for making breathable films, breathable films made at a thickness of 0.05 mm or less are generally flexible, limp, flimsy and hard to handle. When adhesive is applied on the film to enable the film to adhere to the skin, the film tends to stick to itself wherever adhesive surfaces touch each other. This makes it difficult to apply the thin breathable film dressings to the skin.

To overcome this problem, delivery systems have been designed to facilitate handling of these types of dressings. U.S. Pat. Nos. 4,413,621 and 4,485,809 are two examples. One disadvantage of thin breathable film dressings utilizing delivery systems is that it is sometimes difficult for users to figure out how to use them. Another disadvantage of thin breathable film dressings utilizing delivery systems is that it is sometimes difficult for users to actually use the delivery system.

It is also known to use woven fabrics as backings for bandages. Although the woven material itself may be breathable, the finished bandages generally are not breathable after the woven fabric backing has been coated with an adhesive. For example, slot coating an adhesive onto a woven fabric usually completely blocks the openings in the fabric, rendering the bandage non-breathable. To overcome this problem, starve coating techniques have been utilized. The starve coating process involves adjusting the coating die at an angle so that a wave of coating is generated, leaving some area of the bandage un-coated. The lack of an adhesive on some regions of the backing material usually will cause the bandage to curl at the edges, resulting in the bandage falling off.

Therefore, despite the disclosure of the references, there is a continuing need for a non-perforated bandage that is breathable and is easy to handle and apply to a wound.

### SUMMARY OF THE INVENTION

The present invention provides a non-perforated bandage including: a) a woven fabric backing having a first type of yarn and a second type of yarn, the backing having a first major surface and a second major surface; b) a wound contacting pad secured to the first major surface of the backing; and c) a hot melt adhesive, wherein the adhesive is applied to the first major surface of the backing and the bandage is breathable.

### DESCRIPTION OF THE DRAWINGS

Reference is made to the accompanying drawings in which

FIG. 1 is a photomicrograph of the backing fabric used to make the adhesive bandage of the present invention;

FIG. 2 is a photomicrograph of two different yarns of different chemical structures, the uppermost yarn being a polyethylene terephthalate yarn and the lowermost yarn being a polybutylene terephthalate yarn.

FIG. 3 is a photomicrograph of the fabric of FIG. 1 with hot melt adhesive applied thereto in a quantity such that both the warp yarns and the fill yarns carry adhesive; and

FIG. 4 is a photomicrograph similar to FIG. 3 but with a lesser amount of adhesive applied thereto, such that only the warp yarns, but not the fill yarns, carry adhesive.

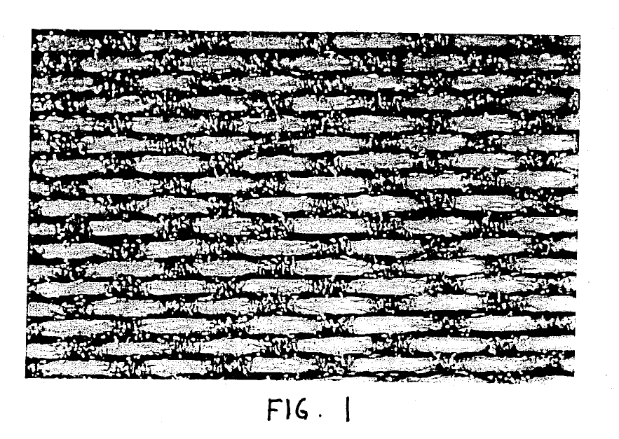

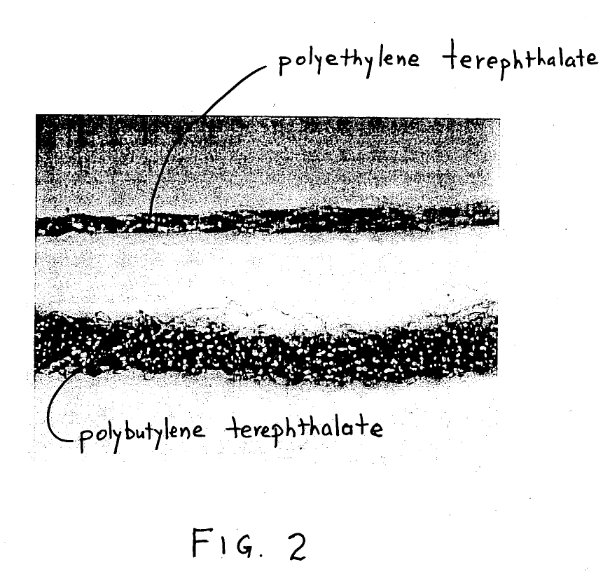

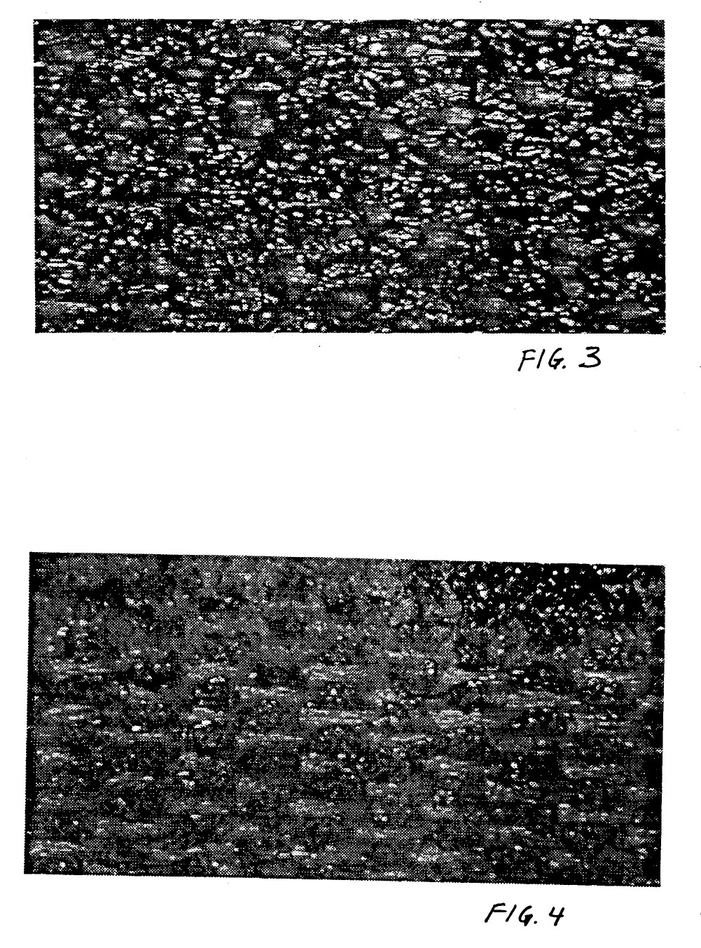

### DETAILED DESCRIPTION OF THE PREFERRED EMBODIMENTS

The backing utilized in the present invention is a woven fabric having a first type of yarn and a second type of yarn. As used herein, by first type of yarn and second type of yarn is meant that the yarns are different in chemical structure.

The woven fabric utilized in the present invention has yarn of one chemical structure in the warp direction and yarn of a different chemical structure in the fill direction. The chemical structure of the first type and the second type of yarn may be selected from cotton, rayon, polyolefins, polyesters, polyamides, and the like. Suitable polyolefins include, but are not limited to, polyethylene and polypropylene. Suitable polyamides include, but are not limited to, nylon 6 and nylon 66. Suitable polyesters include, but are not limited to, poly(ethylene terphthalate) and poly(butylene terephthalate). Those of ordinary skill in the art will recognize that, although only woven fabrics are exemplified in this disclosure, the invention will work as well with knitted fabrics having first and second types of yarns. The term “different chemical structures” includes the case in which, e.g. one of the yarns is nylon and the other of the yarns is polyester. It also includes the case in which both yarns are generically the same (e.g., polyester) and the yarns are different in species. This would be the case, e.g., where one of the yarns is polyethylene terephthalate and the other yarn is polybutylene terephthalate.

The denier (weight of 9000 meters of fiber expressed in grams) of the fibers that make up the woven backing may effect the breathability of the bandage. In general, the lower the denier of the fiber, the higher the amount of adhesive that adheres to the fiber, and therefore the lower the breathability of the bandage. It is therefore possible to combine a fiber with a relatively low denier in one direction (warp or fill), with a fiber of a relatively high denier in the other direction, to obtain a breathable bandage.

The wound contacting pad comprising the bandage is itself breathable and may be made from various materials including rayon; natural fibers, such as, but not limited to, cotton and wood pulp; synthetic fibers, such as, but not limited to, polyester fibers, polyamide fibers, polyolefin fibers, co-polyester fibers, co-polyamide fibers and olefin copolymers fibers; and blends thereof. The fibers may be bicomponent fibers. For example, the fibers may have a core of one polymer, and a sheath of a different polymer. As is known in the art, the fibers may have various deniers and the pad itself may have various basis weights.

The wound-contacting pad is secured to the center region of the adhesive coated first major surface of the woven backing material. In a so-called “strip bandage”, the wound contacting pad is centered from the ends of the bandage and extends from side-to-side of the bandage. In a so-called “island bandage”, the wound contacting pad is centered inwardly of the perimetric edges of the bandage.

Examples of suitable hot melt adhesives which may be utilized in the practice of the invention include, but are not limited to, those based on styrenic block copolymers and tackifying resins such as HL-1491 from HB-Fuller Co. (St. Paul Minn.), H-2543 from ATO-Findley (Wawatausa, Wis.), and 34-5534 from National Starch & Chemical (Bridgewater, N.J.). Ethylene copolymers including ethylene vinyl acetate may also be used. Suitable adhesives also include acrylic based, dextrin based, and urethane based adhesives as well as natural and synthetic elastomers. The adhesives may also include amorphous polyolefins including amorphous polypropylene, such as HL-1308 from HB Fuller or Rextac RT 2373 from Huntsman (Odesssa, Tex.).

The adhesive may be compounded from Kraton® synthetic rubber and the like, or natural rubber with a tackifier, antioxidant, and processing aids. Particularly useful adhesives are disclosed in U.S. Pat. No. 4,080,348, the disclosure of which is hereby incorporated by reference.

A particularly useful rubber based adhesive is that which has a thermoplastic elastomeric component and a resin component. The thermoplastic elastomeric component contains about 55-85 parts of an A-B block copolymer wherein the A blocks are derived from styrene or styrene homologs and the B blocks are derived from isoprene, and about 15-45 parts of a linear or radical A-B-A block copolymer, wherein the A blocks are derived from styrene or styrene homologs and the B blocks are derived from conjugated dienes or lower alkenes, the A blocks in the A-B block copolymer constituting about 10-18 percent by weight of the A-B copolymer and the total A-B and A-B-A copolymers containing about 20 percent or less styrene.

The resin component consists essentially of tackifier resins for the elastomeric component. In general, any compatible conventional tackifier resin or mixture of such resins may be used. These include hydrocarbon resins, rosin and rosin derivatives, polyterpenes and other tackifiers. The adhesive composition contains about 20-300 parts of the resin component per one hundred parts by weight of the thermoplastic elastomeric component. One such rubber based adhesive is commercially available from Ato Findley under the trade name HM3210.

The adhesive can be applied in the molten stage by slot die coating. Slot die coating is a continuous coating process that involves passing the hot melt adhesive through a die onto the backing. Although this process usually results in a continuous coating and a non-breathable bandage, the bandage of the invention is breathable due to the different chemical structure of the yarns comprising the backing material.

The coating weight of the adhesive applied to the backing material effects the breathability of the bandage. The adhesive should be applied to the first major surface of the backing material at from about 20 g/m² to about 80 g/m², preferably from about 50 g/m² to about 70 g/m².

The bandage of the invention may be made in any shape, including, but not limited to, round, oval, rectangular, square, and triangular. The size of the bandage of the invention will vary depending on the desired application. The bandage of the invention may be provided in strip form or in “island pad” form as desired.

The bandage of the invention is useful for many applications, including, but not limited to, bandages and implements for anti-itch agents, acne treating agents, moisturizers, and the like. For bandages, the wound contacting pad thereof may be square, rectangular, round, oval, or triangular in shape. The size of the bandage will depend on the shape of the bandage and the size of the wound meant to be covered by the bandage. Generally, a square bandage may range in size from 5 cm×5 cm to 15 cm×15 cm, preferably from 7.5 cm ×7.5 cm to 12.5 cm×12.5 cm. The length of a rectangular bandage may range from 5 cm to 15 cm, preferably from 7.5 cm to 12.5 cm. The width of a rectangular bandage may range from 0.5 cm to 5 cm, preferably from 1 cm to 3 cm. A circular bandage may range in outer diameter from 5 cm to 20 cm, preferably from 7.5 cm to 17.5 cm, more preferably from 10 cm to 15 cm.

The thickness of the backing material will vary depending on the application, but generally may range from 0.1 mm to 5 mm, preferably 0.5 mm to 3 mm, more preferably 0.5 mm to 2 mm.

The following examples are intended to demonstrate the bandages of this invention. The examples should not be construed as limiting the scope of the invention.

### EXAMPLE 1

Adhesion of Adhesives to Yarns

Yarns of varying chemical structures were tested for adhesion of adhesives to the yarns. In order to form mats of yarn for testing, each yarn to be tested was tightly and uniformly wound on a paper core 21 cm wide. Two to three layers of each yarn were needed to completely cover the core. The cores were taped end to end with masking tape and the yarns were cut with a sharp blade. Masking tape was placed over the cut ends to prevent separation during handling and testing.

The mats of yarns were placed on a work surface and adhesive was applied to the yarns near the taped ends. The purpose of this adhesive was to bind the yarn together and limit separation of the yarn during testing. Each mat was cut in half in the lengthwise direction. Three 2.5 cm×15 cm samples of either HM 3210 hot melt adhesive coated vinyl film or vinyl/acrylic adhesive coated vinyl film were placed lengthwise on the mat, with the adhesive coated side facing the yarn. Each adhesive coated film contained a 1.25 cm tab of masking tape on each side of the film at the end of the film. A 4.55 kg roller was passed over each strip twice.

The adhesive coated mats were tested on a TMI Labmaster Peel Tester, Release and Adhesion Tester, Model 80-90-02-003. The tabs were placed in the jaws of the instrument, and the force required to peel the adhesive film away from the yarn was measured. Each yarn was tested 6 times, and the peel force in g/in was averaged. The results are shown in Table 1.

TABLE 1

Peel Force (g/cm)

| Yarn | Hot Melt Adhesive | Acrylic Adhesive |
|---|---|---|
| Nylon 66 | 1067 | 376 |
| Nylon 6 | 970 | 359 |
| Polyethylene | 784 | 282 |
| Polyethylene/ | 1058 | 313 |
| polypropylene | | |
| Polyethylene | 472 | 165 |
| terephthalate | | |
| Polybutylene | 476 | 176 |
| terephthalate | | |
| Polyethylene | 201 | 74 |
| terephthalate* | | |
| Polybutylene | 454 | 148 |
| terephthalate* | | |

Nylon 6 = Camac 600, available from Camac Company.
Polyethylene = Certran, available through Certran Company.
Polyethylene/polypropylene = polyethylene yarns twisted with polypropylene yarns to provide a twisted yarn
Polyethylene terephthalate = available through Seaway Company.
*= yarn was pre-treated with a dispersion of polytetrafluoroethylene as a waterproofing agent

The data above demonstrates that adhesives adhere differently to yarns based on the chemical structure of the yarns or the surface treatment of the yarns.

### EXAMPLE 2

Adhesion of Adhesives to Yarns Having Different Deniers

Yarns of varying deniers were tested for adhesion of adhesives to the yarns following the same protocol as in Example 1. The hot melt and acrylic adhesives were applied at 37.5 g/m². The results are shown in Table 2.

TABLE 2

| | | Peel Force (g/cm) | |
|---|---|---|---|
| Yarn | Denier | Hot Melt Adhesive | Acrylic Adhesive |
| Nylon 66 | 425 | 1142 | 315 |
| Nylon 66 | 625 | 866 | 197 |
| Nylon 66 | 850 | 472 | 197 |

Denier = total denier of the yarn (as opposed to denier per filament)

The data above demonstrates that adhesives adhere differently to yarns based on the denier of the yarns. As the denier of the yarn increased, the adhesion of the adhesive to the yarn decreased.

### EXAMPLE 3

Breathability of Fabric/Adhesive Laminates

The breathability of woven fabric/hot melt adhesive laminates was tested as a function of the air porosity of the fabric at different hot melt adhesive coating weights. The woven fabric which was used was called “China Flex Fabric”. This is a woven fabric containing different chemical structure yarns in the warp and fill directions, and is available through Hangzhou Limingtou Weaving Co. LTD. The fabric has an unstretched fabric weight of 96 g/m² and a tensile strength of Warp NLT 50 lb/in and fill NLT 25 lb/in. The elongation of the fabric in the Warp direction is NLT 9% and the elongation in the Fill direction is from 40% to 70%. The % recovery in the Fill direction is 70%. The warp yarns comprise about 46 individual polyethylene terephthalate fibers, each such fiber having a denier of about 3, so that the denier of each warp yarn is about 138. The fill yarns comprise about 48 individual polybutylene terephthalate fibers, each such fiber having a denier of about 3, so that the denier of each fill yarn is about 144.

China Flex Fabric was die cut in the machine direction into 7.5 cm×10 cm samples. The samples were coated with different weights of HM-3210 hot melt adhesive using a Nordson melting tank and a Nordson EP51 coating head. The temperature of the adhesive was 350° F. Six samples of each coating weight were analyzed for air porosity on a Gauley Porosity Test Model Category 4301. The results were averaged. The test was performed on China Flex Fabric with and without a coating of a water proofing agent. The results are shown in Table 3.

TABLE 3

| Coating Weight (p/m²) | Porosity (cu.ft./min./sq.ft.) |
|---|---|
| 0 | 0 |
| 37 | 93 |
| 46 | 57 |
| 60 | 22 |
| 70 | 8 |
| 53* | 63 |
| 63* | 48 |
| 77* | 18 |

* = treated with a coating of polytetrafluoroethylene waterproofing agent before adhesive applied

The data above demonstrates that the bandages of the present invention are breathable, however the coating weight of the adhesive effects the breathability of the bandage.

### EXAMPLE 4

Adhesive Bandages

Adhesive bandages are made from the woven fabric/hot melt adhesive laminates of Example 3. The laminated materials of Example 3 are cut into strips approximately 3 inches long and ¾″ wide. Absorbent pads comprising rayon fibers and measuring ¾″ wide and ⅞″ long were placed on the adhesive coated surface of the woven fabric/adhesive laminates to provide strip bandages in which the absorbent pads extend from side-to-side of the bandage and are centered from end-to-end. It will be understood, as is the usual practice, that the adhesive coated areas of the bandages are protected prior to use by release strips.

We claim:

1. A non-perforated bandage comprising:
a) a woven fabric backing having a first type of yarn and a second type of yarn, the backing having a first major surface and a second major surface;
b) a wound contacting pad secured to the first major surface of the backing; and
c) a coating of a hot melt adhesive, wherein the adhesive is applied to the first major surface of the backing and the bandage is breathable.

2. The bandage according to claim 1, wherein the first type of yarn and the second type of yarn are different in chemical structure and are selected from the group consisting of cotton, rayon, polyolefins, polyesters, and polyamides.

3. The bandage according to claim 2, wherein the polyolefins are selected from the group consisting of polyethylene and polypropylene, the polyamides are selected from the group consisting of nylon 6 and nylon 66, and the polyesters are selected from the group consisting of poly(ethylene terphthalate) and poly(butylene terephthalate).

4. The bandage according to claim 1, wherein the adhesive is applied at a coating weight of from about 20 g/m² to about 80 g/m².

5. The bandage according to claim 1, wherein the adhesive is applied at a coating weight of from about 50 g/m² to about 70 g/m².
